(19) United States
(12) Patent Application Publication
(10) Pub. No.: US 2024/0082106 A1
(43) Pub. Date: Mar. 14, 2024

(54) MICROBUBBLE WASHING DEVICE AND MICROBUBBLE WASHING METHOD

(71) Applicant: Thales Inc., Tokyo (JP)

(72) Inventor: Susumu Fujikawa, Tokyo (JP)

(21) Appl. No.: 18/271,357

(22) PCT Filed: Jan. 5, 2022
(86) PCT No.: PCT/JP2022/000125

§ 371 Date: Jul. 7, 2023

(30) Foreign Application Priority Data
Jan. 7, 2021 (JP) 2021-001474

(51) Int. Cl. A61H 33/02; A61H 35/00

(52) U.S. Cl. CPC A61H 33/02; A61H 35/00; A61H 2201/165; A61H 2205/086

(57) ABSTRACT

The present invention provides a microbubble washing apparatus for washing the pubic region and the like using a washing agent composition containing a protease and a lipase, and a washing liquid containing microbubbles and a microbubble washing method using the washing apparatus. The microbubble washing apparatus includes a microbubble generation mechanism configured to prepare washing liquid containing microbubbles, a nozzle configured to discharge the prepared washing liquid, and a washing liquid holding section attachable to cover the pubic region and the like and configured to seal and hold the washing liquid discharged from the nozzle such that the washing liquid does not leak outside. The washing liquid holding section has a washing liquid inlet to introduce the washing liquid discharged from the nozzle into the washing liquid holding section and a washing liquid outlet to eject the introduced washing liquid.

### FIELD OF THE INVENTION

The present invention relates to a microbubble washing apparatus and a microbubble washing method for washing at least one of pubic and anal regions as human body parts, using a washing agent composition containing a protease and a lipase, and a washing liquid containing microbubbles.

### DESCRIPTION OF RELATED ART

In recent years, Japan's population has been aging, and the number of elderly people receiving nursing care, such as the bedridden elderly, has been increasing. In the actual nursing care field, assistance during meals and treatment of excreta take much time and effort. In particular, one of the problems in nursing care for the elderly who wear diapers is that treatment of excreta takes time and effort. Usually, the treatment of excreta adhering to the skin during diaper changes is performed by wiping it off with paper towels or by spraying a small amount of warm water or the like and wiping it off.

However, it is difficult to completely remove excreta with these methods. In addition, the long interval between diaper changes due to lack of manpower may cause skin rash or excreta-related infectious disease. Women, in particular, are at high risk of infectious disease in terms of human anatomy, and even deaths from infectious disease have been reported.

There is a demand for an easier and more sanitary method to be able to perform treatment of excreta for bedridden elderly and sick people.

In connection with the present invention, Patent Literature 1 describes a microbubble washing composition for washing a human body or an animal, characterized by containing a protease and a lipase, a microbubble washing method and a microbubble washing apparatus for washing a human body or an animal, characterized by using a washing liquid containing the washing composition and microbubbles, as well as a microbubble washing method for washing a human body or an animal, characterized by washing using a washing agent composition containing a protease and a lipase, and a washing liquid containing microbubbles.

Patent Literature 2 describes an excrement receiving unit for nursing care that can eliminate discomfort, pain, and other problems that women are particularly prone to feel, and a nursing care garter for attaching the same.

### CITATION LIST

### Patent Literature

Patent Literature 1: JP-A-2008-94726 (US2009214512A1)
Patent Literature 2: JP-A-2019-76342

### SUMMARY OF THE INVENTION

### Problem to be Solved by the Invention

As described above, no method other than wiping off has been considered for treatment of excreta adhering to the skin during diaper changes. Even in the case of tableware, merely wiping off cannot completely remove contamination, and washing away using running water is a common practice. Although whole-body bathing in a bath room can remove waste, bathing requires much time and effort, and many people have difficulty in bathing.

The present invention is made in view of the above circumstances, and an object of the present invention is to provide a microbubble washing apparatus that can wash at least one of the pubic and anal regions of bedridden elderly and sick people in a simple and sanitary manner while their bodies are lying on beds or mattresses, can wash away waste to reduce the risk of infectious disease, and can prevent diaper rash, and to a microbubble washing method using this washing apparatus.

### Means for Solving Problem

The inventor of the present invention have conducted extensive studies in order to achieve the above object and found that by using a microbubble washing apparatus using a washing agent composition containing a protease and a lipase, and a washing liquid containing microbubbles, at least one of the pubic and anal regions of bedridden elderly and sick people can be washed in a simple and sanitary manner while their bodies are lying on beds or mattresses, and waste can be washed away thereby reducing the risk of infectious disease. This finding has led to completion of the present invention.

The present invention provides a microbubble washing apparatus according to the following (1) to (8), and a microbubble washing method according to (9).

(1) A microbubble washing apparatus for washing at least one of pubic and anal regions, using a washing agent composition containing a protease and a lipase, and a washing liquid containing microbubbles, the microbubble washing apparatus comprising:
a microbubble generation mechanism configured to introduce microbubbles into an aqueous solution of the washing agent composition to prepare a washing liquid containing microbubbles;
a nozzle configured to discharge the washing liquid prepared by the microbubble generation mechanism; and
a washing liquid holding section attachable to cover the pubic and anal regions and configured to seal and hold the washing liquid discharged from the nozzle such that the washing liquid does not leak outside, the washing liquid holding section having a washing liquid inlet to introduce the washing liquid discharged from the nozzle into the washing liquid holding section and a washing liquid outlet to eject the introduced washing liquid.

(2) The microbubble washing apparatus according to (1), wherein at least one of pubic and anal regions of a bedridden elderly or sick person is washed.

(3) The microbubble washing apparatus according to (1) or (2), wherein the washing liquid holding section is a waterproof cover covering the pubic and anal regions, or a waterproof pants-like product.

(4) The microbubble washing apparatus according to any one of (1) to (3), wherein the nozzle is inserted into the washing liquid holding section in an upper body direction of a person to be washed, and the washing liquid discharged from the nozzle is introduced into the washing liquid holding section.

(5) The microbubble washing apparatus according to any one of (1) to (4), wherein the washing liquid is prepared by adding the washing agent composition to warm water at 37 to 41° C.

(6) The microbubble washing apparatus according to any one of (1) to (5), wherein the washing liquid is prepared by adding the washing agent composition to warm water at 37 to 41° C. in an amount of 1 to 10 g per 10 liters of the warm water.

(7) The microbubble washing apparatus according to any one of (1) to (6), wherein the washing agent composition used further contains at least one alkali metal salt selected from the group consisting of alkali metal carbonates, alkali metal hydrogen carbonates, alkali metal sulfates, alkali metal halides, and alkali metal salts of boron.

(8) The microbubble washing apparatus according to any one of (1) to (7), wherein a residence time of the washing liquid in the washing liquid holding section is 1 to 10 minutes.

(9) A microbubble washing method for washing at least one of pubic and anal regions of a person in a supine position, using the microbubble washing apparatus according to any one of (1) to (8).

### Advantageous Effects of the Invention

The microbubble washing apparatus using a washing agent composition containing a protease and a lipase, and a washing liquid containing microbubbles, and the microbubble washing method using the microbubble washing apparatus according to the present invention can wash at least one of the pubic and anal regions of a human body in a simple and sanitary manner while the body is lying on a bed or mattress, can wash away waste, and even can reduce the risk of infectious disease.

The washing apparatus and the washing method according to the present invention using a washing agent composition containing a protease and a lipase, and a washing liquid containing microbubbles are gentle to human bodies (especially the sensitive local and anal regions) with low irritation to human bodies and with no need for rubbing.

The microbubble washing method according to the present invention can prevent diaper rash and improve the symptoms for diaper users such as sick people, bedridden elderly people, and babies. It can also prevent bedsore and improve the symptoms.

### BRIEF DESCRIPTION OF THE DRAWINGS

FIG. 1 is a diagram illustrating an exemplary microbubble washing apparatus according to the present invention.

FIG. 2 is a diagram illustrating an exemplary washing liquid holding section with a nozzle attached to a washing liquid inlet in the microbubble washing apparatus according to the present invention.

FIG. 3 is a diagram illustrating an exemplary washing liquid holding section of the microbubble washing apparatus according to the present invention.

FIG. 4 is a diagram illustrating a procedure of attaching the washing liquid holding section illustrated in FIG. 3 to a human body.

FIG. 5 is a diagram illustrating a procedure of attaching the washing liquid holding section illustrated in FIG. 3 to a human body.

### BEST MODE FOR CARRYING OUT THE INVENTION

The present invention provides a microbubble washing apparatus that washes at least one of pubic and anal regions (which hereinafter may be referred to as “pubic region and the like”) using a washing agent composition containing a protease and a lipase, and a washing liquid containing microbubbles (which hereinafter may be referred to as “washing liquid”), and a microbubble washing method for washing at least one of pubic and anal regions of a person in a supine position using this washing apparatus.

Here, “person in a supine person” means a person lying on his/her back.

A microbubble washing apparatus and a microbubble washing method according to the present invention will be described in detail below.

The microbubble washing apparatus according to the present invention can wash the pubic region and the like, specifically, at least one of the pubic and anal regions, preferably the pubic and anal regions, of a person who has difficulty in getting up to go to the toilet or bathroom, such as the bedridden elderly, sick person, and baby, in a simple and sanitary manner while the body is lying on a bed, mattress, or the like, and can wash away waste to reduce the risk of infectious disease.

(Washing Agent Composition)

In the present invention, a washing agent composition containing a protease and a lipase is used. By using such a washing agent composition, release of excreta adhering to the skin surface is facilitated by the protease and lipase, and the excreta can be easily removed and washed away.

The protease refers to a generic name of enzymes catalyzing hydrolysis of peptide bonds.

The protease has a specific optimum pH, and may be classified as an acidic proteinase, a neutral proteinase, or an alkaline proteinase. In the present invention, any of these proteases may be used without specific limitations.

The protease is not particularly limited, but examples thereof include chymotrypsin, subtilisin, pepsin, cathepsin D, thermolysin, papain, caspase, bromelain, actinidin, ficin, trypsin, pancreatin, can be given.

These proteases may be used either individually or in combination of two or more.

As specific examples of the protease, Protin AC10F (manufactured by Daiwa Kasei K.K.), Savinase, Alcalase, Esperase, Durazyme (manufactured by Novozymes), Maxapem, Maxatase (manufactured by Gist-brocades), Bioplaze (manufactured by Nagase Biochemicals, Ltd.), can be given.

It is preferable to use at least pancreatin in order to obtain an excellent washing effect. It is more preferable to use pancreatin and other proteases in combination.

The amount of protease used is not particularly limited. The protease is preferably used in an amount of 0.01 to 0.5 wt %, and more preferably 0.2 to 0.3 wt % based on the total amount of the composition.

The lipase used in the washing agent composition is an enzyme which hydrolyzes fats into glycerol and a fatty acid.

The lipase is not particularly limited, but examples thereof include a lipase from Rhizopus arrhizus, a lipase from Aspergillus niger, and a lipase from a mold such as Rhizopus delemar; a lipase from yeast such as Candida cylindracea; a lipase from bacteria such as Pseudomonas; a lipase which exists in the digestive tract tissue of a ruminant during a lactation period such as pregastric lipase or oral lipase; pig liver lipase; can be given.

These lipases may be used either individually or in combination of two or more. It is preferable to use two or more types of lipases in order to obtain a more excellent washing effect.

Specific examples of the lipase include Sumizyme NLS (manufactured by Shin-Nihon Kagaku Kogyo, Co., Ltd.), Lipase M “Amano” 10, Lipase M “Amano” 10, Lipase G “Amano” 50, Lipase F-AP15, Lipase AY “Amano” 30G, Lipase R “Amano” G, Lipase T “Amano”, Lipase MER “Amano” (manufactured by Amano Enzyme Inc.), Picantase R8000, Picantase A (manufactured by Robin), Toyozyme and LIP (manufactured by Toyobo Co., Ltd.), Lilipase A-10FG, Lilipase AF-5 (manufactured by Nagase ChemteX Corporation), Grindamyl EXEL639 (manufactured by Danisco Culter Japan), Clear-Lens Lipo, Liporaze, Lipex, Lipozyme, Resinase, Paratase, Greasex, Lipopan, Novozyme 435, Lecitase (manufactured by Novozymes), Lipase MY, Lipase OF, Lipase PL, Lipase QLM, Lipase AL, Phospholipase D (manufactured by Meito Sangyo Co., Ltd.), Enzylon AKG (manufactured by Rakuto Kasei Industrial Co., Ltd.), Phospholipase A1 (manufactured by Sankyo Lifetech Co., Ltd.), Lipomod 699L, and Lysomax PF (manufactured by Genencor Kyowa Co., Ltd.).

The lipase used is usually used in an amount of 0.1 to 1.0 wt %, and preferably 0.3 to 0.8 wt % based on the total amount of the composition.

It is preferable that the washing agent composition does not contain a surfactant in order to achieve a more excellent washing effect of microbubbles.

The use of a washing agent composition containing a surfactant may result in excessive foaming, which in turn may reduce the washing effect. When a washing agent composition containing a surfactant is used, a rinsing operation is necessary.

On the other hand, when a washing agent composition containing no surfactant is used, foaming does not occur and rinsing is virtually unnecessary. Further, when a washing agent composition containing no surfactant is used, irritation to humans is little, so there is no problem with touching it, ensuring safe use.

It is preferable that the washing agent composition further includes an alkali metal salt in addition to the protease and the lipase.

The alkali metal salt improves the washing effect, softens water (warm water), and functions as a filler.

The alkali metal salt used is not particularly limited, but examples thereof include

alkali metal carbonates such as sodium carbonate and potassium carbonate; alkali metal hydrogen carbonates such as sodium hydrogen carbonate and potassium hydrogen carbonate; alkali metal sulfates such as sodium sulfate and potassium sulfate; alkali metal halides such as sodium chloride and potassium chloride; alkali metal salts of boron such as sodium tetraborate (borax) and sodium borate; alkali metal silicates such as sodium silicate; alkali metal sulfides such as sodium sulfide; alkali metal nitrates such as sodium nitrate; alkali metal phosphates such as sodium phosphate and sodium dihydrogen phosphate; thiosulfates of an alkali metal such as sodium thiosulfate; can be given.

These alkali metal salts may be used either individually or in combination of two or more.

It is preferable that the washing agent composition used according to the present invention includes at least one alkali metal salt selected from the group consisting of the alkali metal carbonates, the alkali metal hydrogen carbonates, the alkali metal sulfates, the alkali metal halides, and the alkali metal salts of boron. It is more preferable that the composition includes at least one alkali metal salt selected from the group consisting of sodium hydrogen carbonate, sodium sulfate, and borax. It is particularly preferable that the composition includes sodium hydrogen carbonate, sodium sulfate, and borax.

When the alkali metal salt is used, the amount used is usually 80 to 99.5 wt %, and preferably 95 to 99 wt % based on the total amount of the washing agent composition.

When the washing agent composition includes sodium hydrogen carbonate, sodium sulfate, and borax, sodium hydrogen carbonate is preferably used in an amount of 10 to 35 wt %, and particularly preferably 20 to 30 wt % based on the total amount of the composition, sodium sulfate is preferably used in an amount of 30 to 70 wt %, and particularly preferably 40 to 60 wt % based on the total amount of the washing agent composition, and borax is preferably used in an amount of 10 to 35 wt %, and particularly preferably 20 to 30 wt % based on the total amount of the washing agent composition.

It is preferable that the washing agent composition further includes a silica-based desiccant.

The desiccant stabilizes the potencies of the protease and the lipase. As examples of the silica-based desiccant, Sylysia (porous fine silica powder) can be given.

When the silica-based desiccant is used, the amount used is usually 0.01 to 1 wt %, and preferably 0.1 to 0.2 wt %, based on the total amount of the washing agent composition.

If necessary, other components may be further added to the washing agent composition. Examples of other components include inorganic salts other than the above-mentioned alkali metal salts, a perfume, an antiseptic agent, an antibacterial agent, a therapeutic component, and other additives usually used in a washing agent.

As examples of the inorganic salts other than the alkali metal salts, magnesium salts such as magnesium carbonate and magnesium sulfate; calcium salts such as calcium carbonate, calcium nitrate, calcium thiosulfate, and calcium hydrogen phosphate; alum; can be given. The amount of inorganic salts used may be appropriately determined insofar as the effects of the washing agent composition are not impaired.

Examples of the perfume include essential oils such as abies oil, angelica oil, anise oil, copaiba balsam, basil oil, bay oil, bergamot oil, birch oil, rosewood oil, cajabute oil, cassia oil, acacia oil, cedar wood oil, chamomile oil, cinnamon oil, cinnamon leaf oil, citronella oil, elemi oil, eucalyptus oil, geranium oil, white-cedar leaf oil, cypress oil, lavandin oil, lavender oil, lemon oil, mint oil, neroli oil, nutmeg oil, oakmoss oil, ocotea oil, patchouli oil, palmarosa oil, plai oil, rose oil, rosemary oil, sandalwood oil, vetiver oil, and ylang ylang oil; synthetic perfumes such as synthetic musk such as limonene, terpinolene, p-cymene, 9-decenol, mugol, myrcenol, borneol, vetiverol, t-butylcyclohexanol, anisole, anethole, safrole, citral, citronellal, cinnamaldehyde, anise aldehyde, cyclamen aldehyde, citral, acetophenone, benzophenone, acetonaphthone, nerone, and nitromusk, geranyl acetate, bornyl acetate, phenylethyl acetate, myrcenyl acetate, methyl benzoate, methyl cinnamate, methyl anthranilate, synthetic oakmoss, and coumarin.

Examples of the antiseptic agent include sodium benzoate, methyl p-hydroxybenzoate, ethyl p-hydroxybenzoate, propyl p-hydroxybenzoate, butyl p-hydroxybenzoate, isobutyl p-hydroxybenzoate, benzyl alcohol, and benzalkonium chloride.

When other components are used, the amount used is usually 0 to 0.5 wt %, and preferably 0.1 to 0.3 wt % based on the total amount of the washing agent composition.

The method of producing the washing agent composition is not particularly limited. For example, a method may be employed in which a mixture of the protease, the lipase, and other optional components when needed is uniformly mixed and stirred using a known stirrer such as a Vert-o-Mix, a Nauta mixer, a universal mixer/stirrer, a ribbon mixer, a V-shaped mixer, or the like to prepare a powder.

(Washing Liquid)

In the present invention, an aqueous solution containing a washing agent composition containing a protease and a lipase (which hereinafter may be referred to as “washing agent composition”) and microbubbles is used as a washing liquid.

By using the washing liquid containing the washing agent composition and microbubbles, the waste adhering to the pubic region and/or the anal region can be removed reliably in a short time.

The washing liquid can be prepared by introducing microbubbles into an aqueous solution of the washing agent composition.

The aqueous solution of the washing agent composition can be prepared, for example, by filling a solution tank with warm water (37 to 41° C.), adding a predetermined amount of the washing agent composition into the warm water, and lightly stirring the mixture.

The amount of washing agent composition used in the preparation of the aqueous solution of the washing agent composition is usually 1 to 10 g, preferably 3 to 7 g per 10 liters of water. By using the washing agent composition in this range, an excellent washing effect can be achieved.

In order to obtain the washing liquid containing the microbubbles, a microbubble generation device described later is usually used. Since water has a high surface tension, it is impossible to produce bubbles with a diameter of 100 μm or less by normal bubbling.

The microbubbles are minute bubbles with a diameter of 50 μm or less, preferably 5 to 40 μm, and more preferably 10 to 30 μm. Unlike normal bubbles, microbubbles have the property of shrinking and disappearing (crushing) in water. The microbubbles can remove waste by impact waves generated when they disappear(crush). The microbubbles also have the property of carrying negative charge. The microbubbles carrying negative charge disappear in water while bonding to dirt carrying positive charge and floating, thereby causing the dirt to float.

The washing liquid containing the microbubbles is filled with minute bubbles (microbubbles) and becomes clouded. Since the microbubbles have a small buoyancy, the microbubbles float in the water for a long period of time without immediately reaching the water surface, whereby the effects are maintained.

(Microbubble Washing Apparatus)

The microbubble washing apparatus according to the present invention includes a microbubble generation mechanism that prepares a washing liquid, a nozzle that discharges the washing liquid prepared by the microbubble generation mechanism, and a washing liquid holding section having a washing liquid inlet to introduce the washing liquid discharged from the nozzle into the washing liquid holding section and a washing liquid outlet to eject the introduced washing liquid. The washing liquid holding section seals and holds the washing liquid discharged from the nozzle such that the washing liquid does not leak outside.

The microbubble washing apparatus is not particularly limited as long as the microbubble washing apparatus uses a washing agent composition and a washing liquid containing microbubbles to wash the pubic region and the like and includes a microbubble generation mechanism that prepares a washing liquid, a nozzle that discharges the washing liquid, and a washing liquid holding section having a washing liquid inlet to introduce the washing liquid discharged from the nozzle into the washing liquid holding section and a washing liquid outlet to eject the introduced washing liquid, and the washing liquid holding section seals and holds the washing liquid discharged from the nozzle such that the washing liquid does not leak outside.

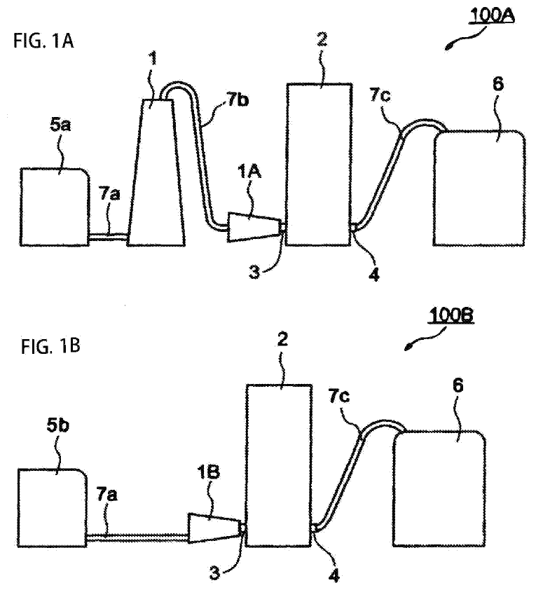

Examples of the microbubble washing apparatus according to the present invention include microbubble washing apparatuses as illustrated in FIGS. 1(A) and 1(B).

A washing apparatus 100A illustrated in FIG. 1(A) includes a microbubble generation mechanism 1, a nozzle 1A, and a washing liquid holding section 2 that seals and holds the washing liquid discharged from the nozzle such that the washing liquid does not leak outside.

The microbubble washing apparatus illustrated in FIG. 1(A) stores an aqueous solution obtained by adding a predetermined amount of washing agent composition to clean warm water (37 to 41° C.) in a container 5a such as a tank. This solution is introduced into the microbubble generation mechanism 1 (microbubble generation device) through a hose 7a using, for example, a pump, and microbubbles are introduced into the solution in the mechanism. The washing liquid containing microbubbles is discharged from a nozzle installed at a washing liquid inlet 3 of the washing liquid holding section 2 to wash the pubic region and the like.

A washing apparatus 100B illustrated in FIG. 1(B) includes a nozzle 1B with a microbubble generation mechanism and a washing liquid holding section 2 that seals and holds the washing liquid discharged from the nozzle such that the washing liquid does not leak outside.

The microbubble washing apparatus 100B illustrated in FIG. 1(B) stores a solution obtained by adding a predetermined amount of washing agent composition to clean warm water (37 to 41° C.) in a container 5b such as a tank with a pressurizing mechanism. This solution is introduced into the nozzle 1B (corresponding to the microbubble generation mechanism 1) under pressure, microbubbles generated in the nozzle 1B are introduced into the washing liquid, and the resulting washing liquid containing microbubbles is discharged from the washing liquid inlet 3 of the washing liquid holding section 2 to wash the pubic region and the like. In the microbubble washing apparatus 100B, a pressurizing device may be disposed separately from the container 5b.

In the microbubble washing apparatus according to the present invention, a heater unit may be provided in the microbubble generation mechanism 1 or at an appropriate location in the solution tank. The heater unit heats the washing liquid so that the temperature of the washing liquid discharged from the washing liquid inlet 3 can be regulated to the set appropriate temperature.

(Microbubble Generation Mechanism)

The microbubble generation mechanism is not particularly restricted insofar as the mechanism can produce microbubbles. A known microbubble generation mechanism may be used. Examples of the microbubble generation mechanism include a device which produces microbubbles in a venturi tube (tube with a narrow constriction) or a discharge port including an orifice plate (doughnut plate with a center opening) (e.g., JP-A-2006-116518 and Japanese Patent Application No. 2006-77553); a device which produces microbubbles by injecting a liquid mixed with a gas into a main pipe disposed in a liquid and causing the liquid to collide with a collision wall disposed downstream of the main pipe (JP-A-2005-334869); a device which produces microbubbles by injecting pressurized air into water through a mesh member or a porous plate with a minute pore size using a pressurized air supply source such as an air pump (JP-A-2006-68631); a device which produces microbubbles by producing a whirling water stream and shearing air using the water stream (JP-A-2003-126665); a microbubble generator described in JP-A-2008-94726; and the like.

(Nozzle)

The microbubble washing apparatus according to the present invention includes a nozzle that discharges the washing liquid prepared by the microbubble generation mechanism.

The nozzle is not particularly limited insofar as the nozzle can discharge a washing liquid containing microbubbles, and a commercially available nozzle can be used.

The microbubble washing apparatus according to the present invention can use a nozzle having a microbubble generation function, for example, as described in Japanese Patent No. 5101391 (a microbubble generation nozzle in which pressurized hot water is passed through an aperture to reduce pressure, causing gases dissolved in the hot water to appear as microbubbles). It is preferable to use the nozzle described in this literature because if so, a compact and lightweight microbubble washing apparatus is provided.

(Washing Liquid Holding Section)

The microbubble washing apparatus according to the present invention includes a washing liquid holding section that seals and holds the washing liquid discharged from the nozzle such that the washing liquid does not leak outside. The washing liquid holding section 2 has a washing liquid inlet to introduce the washing liquid discharged from the nozzle into the washing liquid holding section and a washing liquid outlet to eject the introduced washing liquid.

The washing liquid holding section 2 is attached to cover the pubic and anal regions during use and may have any shape as long as the washing liquid discharged from the nozzle can stay inside for a certain period of time without leaking outside, and the shape and the like are not particularly restricted. Examples include a waterproof cover that covers the pubic and anal regions and a waterproof pants-like product. The waterproof cover and the pants-like product may be made of any material that is waterproof and skin-friendly. For example, materials used for commercially available wet suits can be used.

The waterproof cover that covers the pubic and anal regions and the waterproof pants-like product may be used according to the size (the dimensions such as height and girth) of the person wearing it. For example, small, medium, and large sizes may be used depending on the subject's physique, or a custom-made one may be used to fit the dimensions of the person wearing it.

The washing liquid holding section has a washing liquid inlet to introduce the washing liquid discharged from the nozzle into the interior.

Since bedridden elderly and sick people usually lie on their backs, it is preferable that the washing liquid inlet is provided slightly below the pubic region on the pubic-side face of the person to be washed in order to smoothly perform washing in this state.

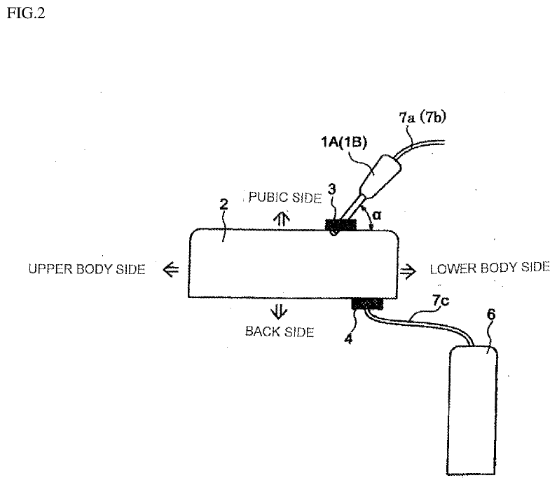

A nozzle that discharges the washing liquid into the washing liquid holding section is installed at the washing liquid inlet. The orientation in which the nozzle is attached is not particularly limited as long as the nozzle installed has an attachable size. In the present invention, in order to spread the washing liquid uniformly to the pubic and anal regions and achieve a sufficient washing effect, it is preferable that the orientation of the nozzle (the direction in which the distal end of the nozzle is inserted into the washing liquid inlet) is such that the nozzle is inserted into the washing liquid inlet toward the upper body direction of the person to be washed. In other words, as illustrated in FIG. 2, it is preferable that the distal end of the nozzle 1A (1B) is inserted into the washing liquid inlet 3 toward the upper body direction. In this case, an angle α between the horizontal direction (a surface of the washing liquid holding section) and the inclination at which the nozzle is inserted is preferably 10 to 70°, more preferably 20 to 60°, and even more preferably 30 to 50° in order to achieve a more excellent washing effect.

The washing liquid holding section has a washing liquid outlet 4. In order to spread the washing liquid to the entire washing liquid holding section and promptly eject the washing liquid containing dirt, it is preferable that the washing liquid outlet 4 is provided slightly below the anal region (the lower body direction of the person to be washed) on the back-side face of the person to be washed.

An ejection pipe connecting to a waste tank is installed at the washing liquid outlet. The washing liquid outlet has a cock to be able to control the retention and ejection of the washing liquid in the washing liquid holding section.

It is preferable that pads are installed inside the washing liquid holding section 2 at positions (symmetrical positions) corresponding to the dimples on the buttocks (depressions on the outside of hip joints). When the pads are installed, the buttocks are generally raised upward, enabling more efficient washing of the pubic region and the like.

The pads have any size that is large enough to cover the central portions of the left and right buttocks (the dimples of the buttocks). The thickness of the pads is preferably 10 to 40 mm and more preferably about 15 to 35 mm. The pads may be made of any material that is waterproof at least on the surface and skin-friendly. For example, the same material as wet suits can be used.

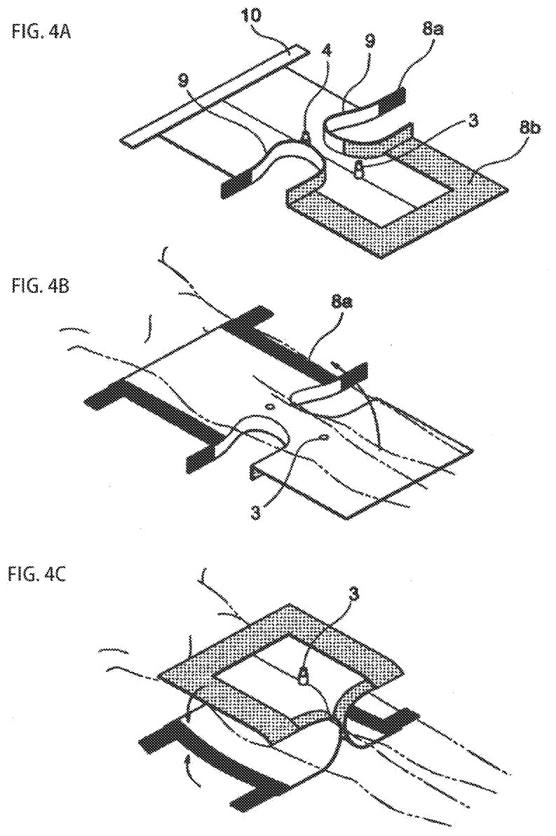

It is also preferable that a pad is installed at a position corresponding to the waist inside the washing liquid holding section 2 (in FIG. 4(A), the inner portion of a waist belt 10). Attaching a pad can more effectively prevent leakage of the washing liquid from the waist side (back side). The thickness of the pad is preferably about 5 mm to 40 mm. The material of the pad is not particularly limited as long as it is waterproof and soft like rubber.

As a method of washing the pubic region and the like using the microbubble washing apparatus according to the present invention, for example, a container such as a tank is filled with a solution obtained by adding a predetermined amount of washing agent composition to clean warm water (37 to 41° C.), subsequently, the solution is introduced into a microbubble generation mechanism (microbubble generation device) by an appropriate method, and microbubbles are introduced into the solution in the mechanism to prepare a washing liquid containing microbubbles. Subsequently, the prepared washing liquid is discharged from a nozzle installed at the washing liquid inlet of the washing liquid holding section to wash the pubic region and the like.

Before washing with the washing liquid, it is desirable to wipe off the excreta adhering to the skin in advance to achieve a more excellent washing effect.

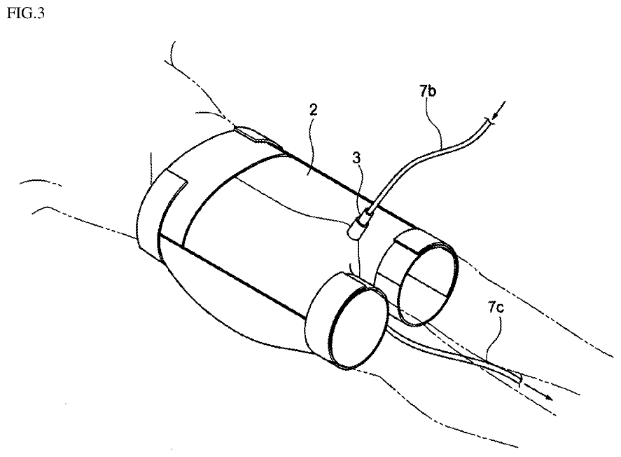

A method of washing the pubic region using the microbubble washing apparatus according to the present invention will be described in more detail, in which the microbubble washing apparatus 100B illustrated in FIG. 1(B) is used and the washing liquid holding section is a ready-to-assemble pants illustrated in FIG. 3 as an example.

The ready-to-assemble pants illustrated in FIG. 3 cover the pubic region and buttocks and are sealed such that water does not leak outside. The washing liquid containing microbubbles is introduced into the washing liquid holding section 2 from the nozzle (not illustrated) installed at the washing liquid inlet 3 through a hose 7b, and stays in the washing liquid holding section 2 for a certain period of time to wash the pubic region and the like. After washing, the washing liquid is sent from the washing liquid outlet (not illustrated) to the waste tank (not illustrated) through a hose 7c.

(1) First, the ready-to-assemble pants illustrated in FIG. 3 (corresponding to the washing liquid holding section of the microbubble washing apparatus according to the present invention) are attached to the person to be washed.

As illustrated in FIG. 4(A), the pants before assembly are prepared. The material of the pants is a waterproof fabric. The waterproof fabric is not particularly limited as long as it is waterproof and skin-friendly. Examples include materials of wet suits.

In FIG. 4(A), numeral 3 denotes the washing liquid inlet, numeral 4 denotes the washing liquid outlet, numeral 8a denotes a hook and loop fastener (male), numeral 8b denotes a hook and loop fastener (female), numeral 9 denotes belts to be wrapped around both legs, and numeral 10 denotes a belt to be wrapped around the waist.

(2) Next, as illustrated in FIG. 4(B), the ready-to-assemble pants illustrated in FIG. 3(A) are placed under the buttocks of the person to be washed.

(3) As illustrated in FIG. 4(C), the fabric on the side having the washing liquid inlet 3 is folded to the pubic side of the human body, like a diaper, and both sides are tightly sealed with the hook and loop fasteners 8a and 8b.

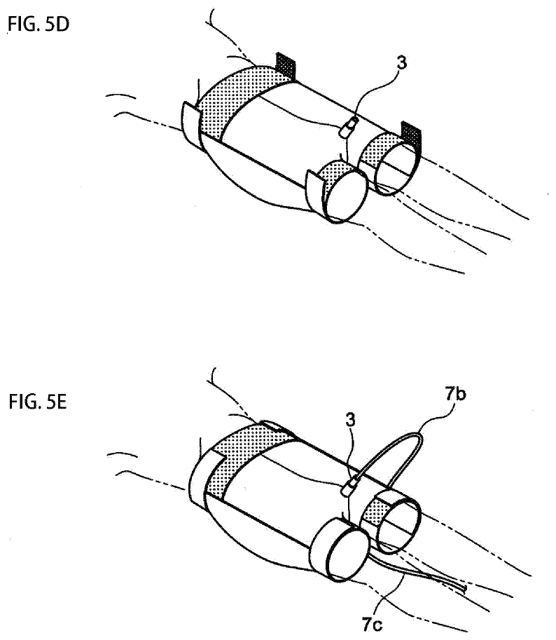

(4) Subsequently, as illustrated in FIG. 5(D), the waist and thighs parts are tightly sealed with the hook and loop fasteners to complete the attachment of the washing liquid holding section. The pants illustrated in the drawings allow the tightness of the belt to be adjusted according to the person's girth and thigh size so that the washing liquid does not leak outside.

(5) Then, as illustrated in FIG. 5(E), the nozzle (not illustrated) connected to the hose 7b is installed on the washing liquid inlet 3, and the hose 7c connected to the waste tank is connected to the washing liquid outlet 4 (not illustrated).

Subsequently, the washing liquid prepared by the microbubble generation mechanism is introduced into the washing liquid holding section through the washing liquid inlet 3.

The amount of washing liquid introduced is about 5 to 15 liters.

The time required for washing (the duration in which the washing liquid stays in the washing liquid holding section) is usually 1 to 10 minutes.

The washing temperature (temperature of the washing liquid) is preferably 37 to 41° C. and more preferably 38 to 40° C. in terms of being gentle to the human body and achieving an excellent washing effects.

After washing, the washing liquid used for washing is ejected from the washing liquid outlet 4. As illustrated in FIGS. 1(A) and 1(B), the washing liquid ejected from the washing liquid outlet 4 can be stored in a waste tank 6 through the hose 7c and disposed of as appropriate. A cock is installed on the washing liquid outlet 4 or the hose 7c. The cock can be opened and closed to control the retention and ejection of the washing liquid. When a cock is installed on the hose 7c, it is preferable that the hose 7c is transparent. This is because if the hose 7c is transparent, the degree of contamination of the washing liquid can be visually checked.

Then, with the cock of the washing liquid outlet 4 kept open, clean washing liquid is introduced into the washing liquid holding section 2 through the washing liquid inlet 3 to lightly wash the pubic region and the like. Such an operation avoids the risk of waste-related infectious disease and eliminates odor problems.

In the washing apparatus and the washing method according to the present invention, rinsing is virtually unnecessary because a washing agent that does not contain a surfactant is used.

Afterwards, the washing liquid holding section attached to the human body can be detached, and the pubic region and the like can be lightly wiped off with a dry towel or the like.

### Example

The present invention will be described in more detail below by way of example, but the present invention is not limited to the following example.

(Example 1)

### (1) Subject

Woman in her 80s (bedridden and in need of nursing care)

Microbubble washing was performed by a female caregiver.

### (2) Washing Agent Composition

The washing agent composition used includes borax: 25 wt %, sodium hydrogen carbonate: 25 wt %, sodium sulfate: 50 wt %, pancreatin: 0.1 wt %, Protin AC: 0.15 wt %, lipase: 0.5 wt %, Sylysia: 0.15 wt %, perfume: 0.2 wt %, and dye: 0.12 wt %.

The components were stirred and mixed by a known stirrer to prepare a washing agent composition.

### (3) Preparation of Washing Liquid

An aqueous solution of the washing agent composition (washing liquid) was prepared by adding 6 g of the washing agent composition to 10 liters of warm water at 40° C. The prepared aqueous solution was stored in a solution storage tank 5b.

(4) Microbubble Washing Apparatus

Ten liters of an aqueous solution of the washing agent composition was used.

The microbubble washing apparatus 100B illustrated in FIG. 1(B) was used.

During use, the distal end of the nozzle 1B was inserted into the washing liquid inlet 3 at an angle of 40° (the angle α in FIG. 2 was 40°) in the upper body direction.

### 5) Microbubble Washing Procedure

The ready-to-assemble pants were attached to the subject according to the procedure in FIGS. 4(A) to 4(C) and 5(D) and 5(E).

Then, as illustrated in FIG. 3, the feed hose 7a of the microbubble washing apparatus 100B was connected to the washing liquid inlet 3, and the feed hose 7c connected to the waste tank 6 was connected to the washing liquid outlet 4.

Subsequently, 10 liters of the aqueous solution of the washing liquid was fed under pressure from the solution storage tank 5b to the nozzle 1B with a microbubble generation function (not illustrated in FIG. 3), and the washing liquid containing microbubbles was discharged from the nozzle 1B into the washing liquid holding section 2 through the washing liquid inlet 3.

Ten liters of the washing liquid was discharged from the nozzle 1B. After five minutes, the cock installed at the washing liquid outlet 4 was opened to eject the washing liquid from the washing liquid holding section 2.

Furthermore, with the cock kept open, another five liters of the washing liquid was discharged from the nozzle 1B into the washing liquid holding section 2 to lightly wash the interior of the washing liquid holding section.

Then, the ready-to-assemble pants were disassembled and the water adhering to the subject's pubic region and the like was wiped off with a towel.

Visual observation showed that the pubic region and the like were clean and no ammonia odor was noticed.

### EXPLANATIONS OF LETTERS OR NUMERALS

1 microbubble generation mechanism
1A nozzle
1B nozzle with microbubble generation function (microbubble generation nozzle)
2 washing liquid holding section
3 washing liquid inlet
4 washing liquid outlet
5a solution storage tank
5b solution storage tank with pressurizing mechanism
6 waste tank
7a, 7b, 7c feed hose
8a hook and loop fastener (male)
8b hook and loop fastener (female)
9 thigh belt
10 waist belt
100A, 100B microbubble washing apparatus

1. A microbubble washing apparatus for washing at least one of pubic and anal regions, using a washing agent composition containing a protease and a lipase, and a washing liquid containing microbubbles, the microbubble washing apparatus comprising:
a microbubble generation mechanism configured to introduce microbubbles into an aqueous solution of the washing agent composition to prepare a washing liquid containing microbubbles;
a nozzle configured to discharge the washing liquid prepared by the microbubble generation mechanism; and
a washing liquid holding section attachable to cover the pubic and anal regions and configured to seal and hold the washing liquid discharged from the nozzle such that the washing liquid does not leak outside, the washing liquid holding section having a washing liquid inlet to introduce the washing liquid discharged from the nozzle into the washing liquid holding section and a washing liquid outlet to eject the introduced washing liquid.

2. The microbubble washing apparatus according to claim 1, wherein at least one of pubic and anal regions of a bedridden elderly or sick person is washed.

3. The microbubble washing apparatus according to claim 1, wherein the washing liquid holding section is a waterproof cover covering the pubic and anal regions, or a waterproof pants-like product.

4. The microbubble washing apparatus according to claim 1, wherein the nozzle is inserted into the washing liquid holding section in an upper body direction of a person to be washed, and the washing liquid discharged from the nozzle is introduced into the washing liquid holding section.

5. The microbubble washing apparatus according to claim 1, wherein the washing liquid is prepared by adding the washing agent composition to warm water at 37 to 41° C.

6. The microbubble washing apparatus according to claim 1, wherein the washing liquid is prepared by adding the washing agent composition to warm water at 37 to 41° C. in an amount of 1 to 10 g per 10 liters of the warm water.

7. The microbubble washing apparatus according to claim 1, wherein the washing agent composition further contains at least one alkali metal salt selected from the group consisting of alkali metal carbonates, alkali metal hydrogen carbonates, alkali metal sulfates, alkali metal halides, and alkali metal salts of boron.

8. The microbubble washing apparatus according to claim 1, wherein a residence time of the washing liquid in the washing liquid holding section is 1 minute or more and 10 minutes or less.

9. A microbubble washing method for washing at least one of pubic and anal regions of a person in a supine position, using the microbubble washing apparatus according to claim 1.
